(19) United States
(12) Patent Application Publication
(10) Pub. No.: US 2021/0043288 A1
(43) Pub. Date: Feb. 11, 2021

(54) Individual Health Information Aggregation System

(71) Applicant: KABUSIKIGAISYA DIGIMED, Wakayamai (JP)

(72) Inventor: Yoshikatsu NAKANISHI, Wakayama (JP)

(21) Appl. No.: 16/979,846

(22) PCT Filed: Feb. 26, 2019
(86) PCT No.: PCT/JP2019/007249

§ 371 Date: Sep. 10, 2020

(30) Foreign Application Priority Data
Apr. 13, 2018 (JP) 2018-077609

(51) Int. Cl. G16H 10/65; G16H 40/20; G16H 40/67; G16H 80/00; G06F 21/32; G06F 21/35

(52) U.S. Cl. CPC G16H 10/65; G16H 40/20; G06F 21/35; G16H 80/00; G06F 21/32; G16H 40/67

(57) ABSTRACT

The present invention provides an individual health information aggregation system for aggregating and managing health information such as medical records and test results of examinees spanning a plurality of medical institutions in an aggregation server of a management center in association with identification information (patient number) unique to each examinee, and enabling treatment and administration of medication to be appropriately carried out as a result of the examinee or a family member or a person connected with a medical institution viewing the heath information as appropriate. Inside of an ambulance, when the examinee is transported in an emergency situation, the system downloads and displays the health information on a screen as a result of a medical professional obtaining the identification information (patient number), and transmitting the identification information to the management center together with his or her own ID and password.

### TECHNICAL FIELD

The present invention relates to a system that is able to aggregate the health information of individuals, particularly health information acquired in a large number of medical institutions (hospitals, etc.) or a plurality of clinical departments in one medical institution.

### BACKGROUND ART

In many cases, when an examinee (patient) develops a condition, he or she seeks consultation at a specific medical institution (his or her family doctor's medical institution or a local medical institution), and, depending on the condition, is referred to another medical institution (large hospital) or a medical institution specializing in a different field. Accordingly, the health information of the examinee, such as examination (diagnostics) results (medical records), test results (biochemical tests, images, etc.) and medication histories (prescriptions), for example, are conventionally held at the medical institution where the consultation took place, thus allowing the examinee to be appropriately cared for in terms of treatment, administration of medication and the like, for example.

However, in cases such as where the cause of the condition is not clear such as with so-called intractable diseases or cases such as where an elderly person is concurrently treated at a number of hospitals, clinical departments or the like, redundant tests, administration of medication and the like are a reoccurring problem. The health information is often shared between multiple clinical departments of the same medical institution over a network within the medical institution. However, across different medical institutions, the health information will be different starting with the identification number (number of the patient registration card, medical record, etc.) of the examinee, and in cases such as where a second opinion is sought, for example, the examinee or a family member will have to borrow the health information from the medical institution where consultation was previously sought and bring the borrowed health information with them. In recent years, health information such as medical records and test results was freely made available upon request by the examinee or a family member, in order to get a second opinion or the like.

In view of this, the inventor of the present invention has previously proposed Patent Document 1. Patent Document 1 is a system for bringing health information borrowed from a medical institution by an examinee or a family member to an aggregation facility and performing aggregation (name-based aggregation) of the health information such that x-ray images, biochemical test results and the like are discernible at a glance. Heretofore, this enabled several tens of CDs worth of health information, for example, provided by various hospitals to be aggregated and readily managed. Specifically, the carrying of multiple pieces of health information (files, CDs, etc. of the x-ray images and biochemical test results) borrowed from different medical institutions is facilitated for examinees and their family, and, on the medical institution side, redundant tests are avoided and developments can be readily confirmed.

Additionally, aggregation of individual health information is also shown in Patent Document 2 and Patent Document 3. Also, testing of health information aggregation by the Ministry of Health, Labour and Welfare has started in some areas as “a demonstration project to build a foundation for utilizing health information directed toward the establishment of local medical cooperation”, and medication information has, furthermore, already been implemented as electronic medication notebooks in many areas, prior to health information in general.

### CITATION LIST

### Patent Document

Patent Document 1: JP 5140310B

Patent Document 2: JP 2011-175373A

Patent Document 3: JP 2001-325372A

### DISCLOSURE OF INVENTION

### Problem to be Solved by the Invention

The abovementioned conventional technology is extremely effective for ensuring that appropriate treatment is implemented through medical institutions sharing the health information of examinees, and for a local insurance society or medical association checking that there is no duplication of medication and no errors in the medical billing claim. Also, troublesome and time-consuming tasks such as getting a referral from the medical institution that the examinee previously visited, in order for the new hospital or clinical department that the examinee seeks consultation to obtain the previous health information of the examinee, can be dispensed with.

However, the health information of each examinee is held in association with unique identification information such as an ID or the like that is allocated in advance for every examinee, and, when required, an examinee or a family member have to present that identification information to medical professional (authorized person). Thus, in cases such as where an examinee visits a medical institution for consultation as an outpatient, he or she can have the identification information ready for presentation together with his or her health insurance card and the like, but in situations such as where the examinee is transported in an emergency situation to a medical institution in an unconscious state, medical professional such as emergency paramedics will be unable to readily acquire such identification information, and the medical institution that is supposed to receive the examinee will be in a similar situation.

### Means for Solving Problem

An object of the present invention is to provide an individual health information aggregation system that is able to effectively utilize aggregated health information of individuals in an emergency.

In order to realize the abovementioned object, an individual health information aggregation system of the present invention, in realizing efficient and appropriate medical treatment by using aggregated health information of individual examinees, acquires the identification information of the examinee with acquisition means, in the place where the examinee is being accommodated such as inside an ambulance, when the examinee is in poor physical condition during transportation in an emergency situation or the like, and the health information of the examinee is displayed on display means when it is authenticated with authentication means that medical professional such as a paramedic has the credentials to access (download) the health information of the examinee.

Therefore, it becomes possible for the health information of individuals that is aggregated and managed to also be used in emergencies, facilitating medical professionals in taking appropriate action, and enabling the survival rate to be improved.

### BRIEF DESCRIPTION OF DRAWINGS

FIG. 1 is a block diagram showing the overall configuration of an individual health information aggregation system according to one embodiment of the present invention, and shows a state where an examinee is transported in an emergency situation.

FIG. 2 is a block diagram showing the overall configuration of an individual health information aggregation system according to one embodiment of the present invention, and shows a state where an examinee in under normal conditions.

FIG. 3 is a diagram showing an example of an entry screen for accessing health information of an examinee in the individual health information aggregation system.

FIG. 4 is a diagram showing an example of an input result and obtained health information.

FIG. 5 is a diagram showing another example of an entry screen for accessing health information of an examinee in the individual health information aggregation system.

FIG. 6 is a diagram showing a transmission image of live images of an examinee in the individual health information aggregation system.

FIG. 7 is a diagram showing a reception image of the live images.

### DESCRIPTION OF THE INVENTION

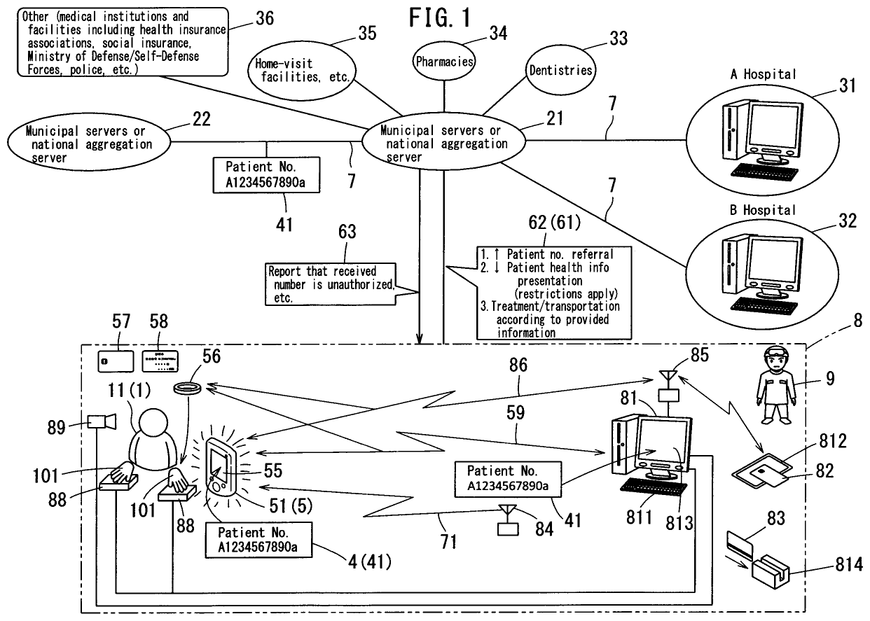

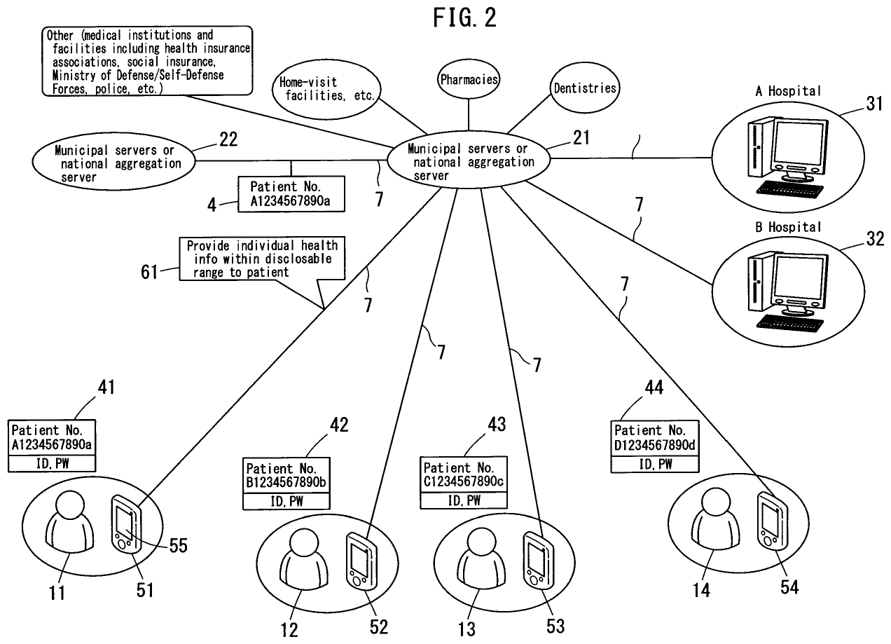

FIG. 1 and FIG. 2 are block diagrams showing the overall configuration of an individual health information aggregation system according to one embodiment of the present invention. FIG. 1 shows the state where an examinee (more appropriately referred to a patient in some cases, but “examinee” will be used hereinafter for uniformity) 1 is transported in an emergency situation, and FIG. 2 shows a state under normal conditions. Referring to FIG. 2, the individual health information aggregation system of the present embodiment aims to enhance convenience, as a result of health information related to individual examinees 11 to 14 (hereinafter, denoted by reference sign 1 when referred to collectively) being aggregated and managed in aggregation servers of management centers 21 and 22 (hereinafter, denoted by reference sign 2 when referred to collectively). The management center 2 need only be determined as appropriate, and may be installed in each administrative district or centrally manage the entire country.

Specifically, this individual health information aggregation system aggregates and holds, for each of a plurality of examinees 1, health information that includes, as appropriate, a current medical record, medication information and the like, and also a past clinical history, chronic conditions and the like for each examinee 1 collected in a plurality of medical institutions 31 to 36 (hereinafter, denoted by reference sign 3 when referred to collectively) in association with identification information 41 to 44 (hereinafter, denoted by reference sign 4 when referred to collectively), which are unique patient numbers assigned in advance to each examinee 1, in the management center 2. This individual health information aggregation system enables health information of each of the examinees 1 to be viewed based on a predetermined rule, in response to a request from medical professional, the examinee 1 or a member of his or her family, or the like. In this individual health information aggregation system, medical test images are, for example, exchanged in the form of data conforming to DICOM (Digital Imaging and Communications in Medicine), which is a standard relating to medical test images and communication thereof, and are also held in the management center 2. In the management center 2, a database that actually holds the health information can be realized by an aggregation server such as NAS (Network Attached Storage).

In the example of FIG. 2, the medical institutions 31 and 32 are general hospitals, the medical institution 33 is a dentistry, the medical institution 34 is a pharmacy, the medical institution 35 is a home-visit nursing care facility, and the medical institution 36 is another healthcare-related facility (health insurance society, social insurance, Ministry of Defense (Self-Defense Force), police force, etc.). FIG. 2 shows an example in which the identification information 41, 42, 43 and 44, namely, “A1234567890a”, “B1234567890b”, “C 1234567890c” and “D 1234567890d”, are respectively allocated to the examinees 11, 12, 13, and 14. The examinees 11 to 14 access the aggregation server of the management center 2 from mobile information terminals 51 to 54 (hereinafter, denoted by reference sign 5 when referred to collectively) such as a smartphone or tablet terminal in their possession, and, through input of the identification information 4 and a predetermined login ID and password, it becomes possible for health information 61 of oneself, a family member, or the like to be viewed on a display screen 55 or presented at the medical institution 3. The medical institution 3, the management center 2, and the mobile information terminal 5 are connected to each other via a network 7.

The individual health information aggregation system of the present invention is thus extremely effective for the examinee 1 or a family member going to a new medical institution (31, 32, 36) as an outpatient taking the health information 61 of the examinee 1 with him or her in order to get a second opinion or the like, and for a local insurance society or medical association which is not shown checking that is no duplication of medication and no errors in the medical billing claim. Also, with this individual health information aggregation system, troublesome and time-consuming tasks such as getting a referral, in order for the other hospital (31, 32, 36) or clinical department to obtain previous health information, can be dispensed with.

Note that the predetermined rule for viewing of the health information 61 is for actions such as restricting the health information 61 to a level viewable by a doctor, to a level viewable by a pharmacy, and to a level viewable by the examinee 1 himself or herself or a family member. Specifically, the rule may be such that all information can be viewed by a clinical department in a hospital (31, 32, 36), but a pharmacy (34) or the examinee 1 is unable to view information relating to the progressive stage, and information on communicable diseases (HIV, hepatitis, etc.) that is required at the time of treatment can only be viewed by a hospital (31, 32, 36). The identification information 4 is an ID number or the like that is allocated one to each examinee, and is not allocated for every medical institution (31, 32, 36) unlike that on a patient registration card, and also differs from the above login ID and password that the examinee 1 is able to arbitrarily set in advance.

The present invention enables the health information 61 is be utilized when the examinee 1 is in poor physical condition, such as when transported in an emergency situation, for example, using the individual health information aggregation system constituted as mentioned above. Referring to FIG. 1, the health information 61 is utilized at the accommodation place 8 of the examinee 1 such as inside an ambulance or the hospital that is the transportation destination. The accommodation place 8 may be the inside of an ambulance, the treatment room of the primary transportation destination in the case where the examinee 1 requires secondary transportation, or a public facility or the like such as a gymnasium or a conference room, in the case where there are a large number of examinees 1 such as in a major disaster.

In the present invention, it is possible to carry out appropriate measures within the accommodation place 8 and advance preparations for receiving the examinee 1 at the transportation destination such as a hospital (31, 32), as a result of a medical professional 9 such as a paramedic, a doctor or a nurse who perform first-aid measures utilizing the health information 61 in the accommodation place 8. More specifically, the identification information 4 of the examinee 1 is first acquired. Acquisition of the identification information 4, in the case where the examinee 1 is conscious and remembers the identification information 4, is carried out by the medical professional 9 asking the examinee 1 and inputting (typing in) the identification information 4 provided by the examinee 1 with a keyboard 811 of a personal computer 81 serving as acquisition means in a manner that will be described later. Alternatively, when the examinee 1 does not remember the identification information 4 or is not conscious, the medical professional 9 himself or herself or the system automatically acquires the identification information 4 in a manner that will be described later, and inputs the acquired identification information 4 into the personal computer 81. At the accommodation place 8, the means of inputting the identification information 4 is not limited to the fixedly installed personal computer 81, and a movable tablet terminal may be used, or both of these means may be used together.

In this individual health information aggregation system, when the identification information 4 is thus obtained, the medical professional 9 accesses the aggregation server of the management center 2, and accesses (downloads) the health information of the examinee 1, as denoted by reference sign 62, with the personal computer 81 serving as authentication means, using his or her own ID, password, or the like. The ID and password are used in authenticating whether the medical professional 9 has the credentials to access (download) the health information 61 of the examinee 1. Input of the ID and password may be performed with the keyboard 811, but may also be carried out by an IC card 82 or a magnetic card 83 that the medical professional 9 possesses being brought close to, in contact with, inserted into a card reader 812 or 814, or the like, and information being read.

In cases such as where the identification information 4 of the examinee 1 is not correct (the corresponding examinee does not exist, i.e., the identification information 4 does not exist), or where the ID or password of the medical professional 9 is not correct, that is, the password is incorrect or has been changed, or when the ID is no longer valid, the aggregation server of the management center 2 sends a reply indicating that this information is incorrect to the personal computer 81, as denoted by reference sign 63. The ID of the medical professional 9 is not applied for individually, for example, but rather the affiliated organization such as the firefighters association collectively applies to a system administrator and receives issuance.

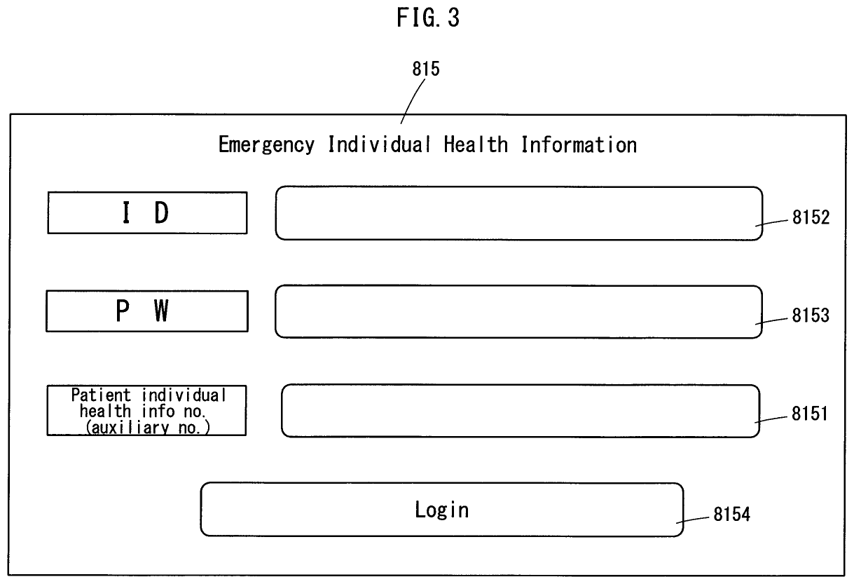

FIG. 3 shows an example of an entry screen 815 of the personal computer 81 at the time of the medical professional 9 accessing (downloading) the health information 61 of the examinee 1. It is possible for the medical professional 9 to access the aggregation server, by entering the identification information 4 into an entry field 8151 as a patient individual health information number, entering his or her own ID into an entry field 8152, entering a password into an entry field 8153, and clicking a login button 8154. In the case of using the IC card 82 and the magnetic card 83, the entry fields 8152 and 8153 will be entered automatically.

When authentication of the medical professional 9 is performed by the aggregation server and the health information 61 of the examinee 1 exists, the health information 61 is downloaded and displayed on a display screen 813 of the personal computer 81 which is display means.

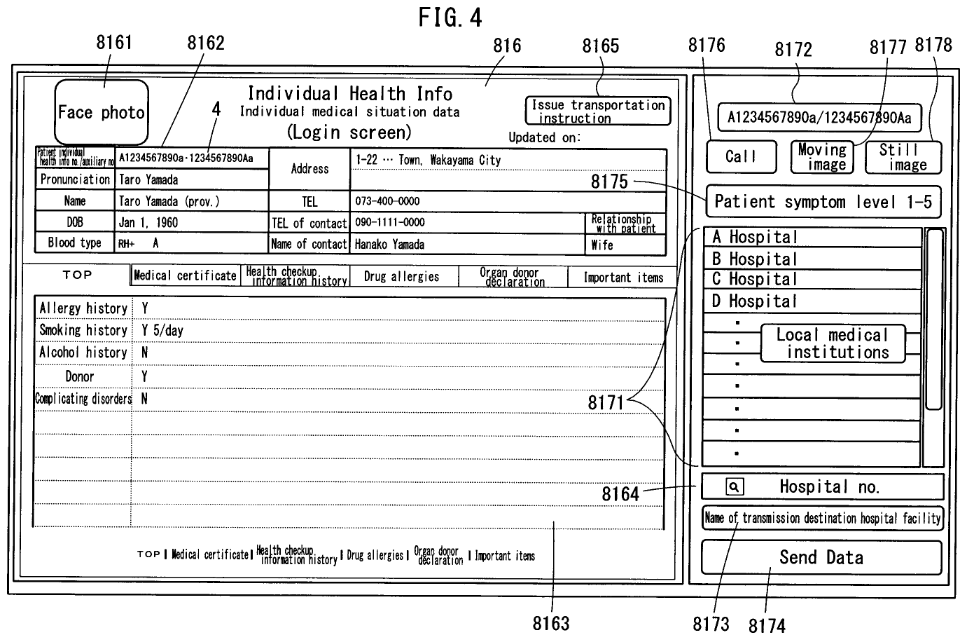

FIG. 4 is a diagram showing an example of a display screen 816. Broadly, a face photo 8161 and personal information 8162 are displayed in upper left fields. Accordingly, personal authentication of the examinee 1 is performed with the identification information 4 as aforementioned, and the medical professional 9 is able to again perform personal identification of the examinee 1 with this face photo 8161. The personal information 8162 includes the identification information 4, name, birth date, blood type, address, telephone number, contact, and the like. An instruction button 8165 is a button for requesting selection of a receiving medical institution by the local fire brigade headquarters (Tel: 119).

A lower left field 8163 of the display screen 816 is in a multiple page configuration, and basic information (TOP) such as allergies and preferences, medical certificates of previous consultations, health checkup results, an organ donation declaration and the like are displayed. Aright-side field 8164 of the display screen 816 is used for coordinating with the medical institution serving as the transportation destination, and includes items used in exchanges for preparing to receive the examinee 1, which will be described below, in addition to medical institutions serving as receiving destination candidates being displayed in candidate order and contact details and the like being displayed.

According to this individual health information aggregation system, this enables the health information 61 of individuals that is aggregated and managed in the management center 2 to also be effectively used in emergencies such as an injury, illness, accident or disaster, facilitating the medical professional 9 in taking appropriate action, and enabling to the survival rate to be increased.

Here, another acquisition method will be specifically described in relation to the identification information 4, using FIG. 1. First, as mentioned above, the examinee 1, at the point of desiring a new consultation as an outpatient or the like (at another hospital, etc.), takes the information communication terminal 5 on which a dedicated application has been installed in advance with him or her, in order to be able to access his or her own health information 61 as appropriate, as shown in FIG. 2. On the information communication terminal 5, it is possible for the examinee 1 to access of his or her own identification information 4 and the health information 61 associated therewith, by being authenticated as an authorized user, through ID and password authentication.

Accordingly, given that such a dedicated application has been installed, the examinee 1, when in poor physical condition (when transported in an emergency situation, etc.), is also able to present this information communication terminal 5, in the case of not remembering the identification information 4, by having the medical professional 9 view the information communication terminal 5. In this case, as the acquisition means, the medical professional 9 need only check the display screen 55 of the information communication terminal 5, thus facilitating acquisition of the identification information 4.

In contrast, in cases where the examinee 1 cannot respond, such as when the examinee 1 is not conscious, the medical professional 9 must perform an operation to start up the information communication terminal 5 and acquire the identification information 4. In this case, given the dedicated application is installed on the information communication terminal 5 in advance, preferably the dedicated application is started up by recognition of a predetermined voice command by the medical professional 9, through speech recognition that utilizes a so-called artificial intelligence (AI) speaker or a search function as a predetermined startup operation, and the identification information 4 is displayed on the display screen 55.

As a result, even if the owner (examinee 1) is in poor condition and does not carry out the complex personal authentication through ID and password entry or the like in person, it is possible for the medical professional 9 to search for the identification information 4 and access the health information 61, thus enabling the medical professional 9 to reliably acquire this identification information 4, even in a situation where the owner (examinee 1) is in poor condition and the identification information 4 cannot be found out directly. Also, the dedicated application does not need to be left open all the time, in which case the power consumption of the information communication terminal 5 can be reduced.

Also, as mentioned above, in the case where the dedicated application is installed on the information communication terminal 5 in advance, preferably the identification information 4 is transmitted to the personal computer 81, which is acquisition means, by predetermined wireless communication such as wireless LAN, as denoted by the reference sign 59, and automatically set. In other words, the entry of the identification information 4 into the entry field 8151 in FIG. 3 is automatic. As a result, reduction of the load on the medical professional 9 can be achieved and the entry time can be shortened.

However, in this case, the wireless communication could possibly leak outside of the accommodation place 8 of the examinee 1 such as an ambulance. When the party that intercepts the wireless communication has means for being able to pass authentication such as the ID, password or the like of the medical professional 9, due to having worked in other medical institutions, for example, health information 61 such as medical records which is personal information of the examinee 1 will be viewable. With regard to this point, as aforementioned, in the case where the medical professional 9 manually enters the identification information 4 displayed on the information communication terminal 5 or found out directly from the examinee 1 into the entry field 8151 of the personal computer 81, which is acquisition means, with the keyboard 811, this identification information 4 will not leak outside of the accommodation place 8 in acquiring (setting) the identification information 4.

As such, the identification information 4 is preferably transmitted between the information communication terminal 5 and the personal computer 81, which is the acquisition means, after being encrypted at a predetermined timing, such as a predetermined cycle, for example, with an encryption sequence that is changed each time. As a result, undesirable leaking of personal information can be prevented. A plurality of encryption sequences may be prepared in the dedicated application and the encryption sequence to be used may be switched in communication from the management center 2 via the network 7, or a new (once only) encryption sequence may be set as appropriate, in communication from the management center 2 via the network 7. Of course, acquisition of the health information 61 via the network 7 and access to the management center 2 denoted by the reference sign 62 are also performed after encryption.

Also, as mentioned above, in the case where the medical professional 9 wants to acquire the identification information 4 by operating the information communication terminal 5 of the examinee 1, the medical professional 9 may not know whether the owner (examinee 1) has an information communication terminal 5 or where the information communication terminal 5 is even if the examinee 1 has one. In such cases, the necessity may arise to ask an examinee 1 who is unconscious. As such, in the present embodiment, a dummy transmitter 84 is provided in the accommodation place 8.

This dummy transmitter 84 is capable of transmitting radio waves via a mobile phone network 71, and an information communication terminals such as a smartphone is normally standing by to receive communication from this network 71. This dummy transmitter 84, in response to the issuance by the medical professional 9, then issues notification of an emergency such as an earthquake in a dummy manner, at a level that an arbitrary information communication terminal 5 in this accommodation place 8 would be capable of receiving, or in other words, at a transmission power low enough to not leak externally. Then, by an information communication terminal 5 that received the notification reacting in a manner indicating that the incoming notification was received, the existence of an information communication terminal 5 can be indicated to the medical professional 9. Accordingly, even in situations such as where the owner (examinee 1) is in poor condition and the whereabouts of mobile information terminal 5 cannot be found out directly, the medical professional 9 is able to reliably acquire the identification information 4.

Also, by increasing the power of the dummy transmitter 84 and installing the dummy transmitter 84 outside, a dummy emergency notification can be issued, when a user gets in distress at a ski field, for example, causing the mobile information terminal 5 to receive the incoming notification, and this notification can be useful in specifying of the location of the user (examinee 1). Thus, the dedicated application software preferably continue issuing sound or light for a while after reception of the emergency notification and/or repeating these operations at a predetermined interval.

Alternatively, instead of the mobile phone network 71, a short-range wireless LAN network 86 such as a so-called Wi-Fi (registered trademark; IEEE-802.11 standard) or Bluetooth (registered trademark, IEEE-802.15 standard) may be used. More specifically, the accommodation place 8 has a wireless LAN spot 85, a predetermined IP address common to the individual health information aggregation system is allocated to the wireless LAN spot 85, and, as with free Wi-Fi, the information communication terminal 5 responds when radio waves are caught, assuming the reception settings have been configured. The dedicated application that is downloaded in advance to the information communication terminal 5 is the same type of software as the wireless LAN spot 85, and is activated by reception of the Wi-Fi radio waves, and the health information 4 of the examinee 1 is transmitted from the wireless LAN spot 85 to the personal computer 81 that is connected thereto. Once the health information 4 is transmitted, the identification information 4 is automatically set in the entry field 8151 of FIG. 3.

The medical professional 9 is also able to reliably acquire the identification information 4 if such a configuration is adopted, even in cases such as where the owner (examinee 1) is in poor condition and the identification information 4 cannot be retrieved by operating the mobile information terminal 5.

Here, as mentioned above, when the identification information 4 is transmitted over free Wi-Fi or the like, the information could possibly be intercepted outside the accommodation place 8. The identification information 4 is extremely important personal information, and there are techniques such as encryption in this case. On the other hand, as aforementioned, the ID and password for the examinee 1 to access his or her own health information 61 are set to in the mobile information terminal 5 which is a smartphone or the like. Accordingly, security when using the short-range wireless LAN network 86 can be improved by using the ID and password.

Specifically, the examinee 1 causes the mobile information terminal 5 to store the ID and password in advance by entering the ID and password into the mobile information terminal 5, similarly to general login ID and password storage. Because user authentication can be performed with the ID and password, a configuration may be adopted in which the health information 61 of the examinee 1 can be similarly acquired in the mobile information terminal 5, using different information from the identification information 4. However, because completely different information could possibly lead to confusion, preferably an auxiliary number obtained by changing part of the identification information 4 is used. For example, in FIG. 1 and FIG. 2, the identification information 41 of the examinee 11 is “A1234567890a” as aforementioned, but the auxiliary number illustrated in FIG. 4 is “1234567890Aa.” As a result, even though viewing of medical records becomes possible, for example, the most important portion in the individual health information such as infection information that can only be viewed in the original identification information 4, can be made confidential. Note that the most important portion in the individual health information is decided by a doctor, in accordance with an online manual for personal information.

By adopting this configuration, the information communication terminal 5 automatically responds to a call from the wireless LAN 86, when the examinee 1 is in poor physical condition, and even if the auxiliary number is intercepted by a third party, the original identification information 4 which is important personal information is not divulged, and the personal information can be protected.

Furthermore, SNS has become widespread, as a configuration in which the information communication terminal 5 stands by and responds after reception. The response currently requires an operation by a user (examinee 1). However, in the present embodiment, as mentioned above, a dedicated application is installed on the information communication terminal 5. As such, a configuration may be adopted in which, using this dedicated application, the medical professional 9 sends an SNS message on LINE (registered trademark), Twitter (registered trademark) or the like, and the application of the information communication terminal 5 is activated by receiving the SNS registered in advance, and sends back the user (examinee 1) name, telephone number, identification information 4 or the like to the personal computer 81, via the network 71 or the wireless LAN 86. As a result, the identification information 4 can be automatically acquired, utilizing the social network standby function of the information communication terminal 5.

On the other hand, a configuration may be adopted in which automatic issuance of the identification information 4 can also be performed by a dedicated device with limited functions, rather than being limited to a general-purpose information communication terminal 5 such as a smartphone. The dedicated device is preferably an accessory 56 that can be naturally worn on the body by the examinee 1 for as long as need be. A bracelet or the like that is habitually worn is given as an example. In this case, a first wireless device of the wireless LAN circuit 86 of the wireless LAN spot 85 or the like installed in the accommodation place 8 or a mobile phone network (71) serves as acquisition means, and the accessory 56 worn by the examinee 1 serves as a second wireless device. The first wireless device of the wireless LAN spot 85 or the like searches (polls, probes) for a responding terminal, while the accommodation place 8 is in use (during transportation in an emergency situation). The accessory 56 is also provided with a storage unit that stores the identification information 4, and sends back the identification information 4 to the personal computer 81 via the mobile phone network (71) or the wireless LAN network 86, in response to the search.

As a result, the first wireless device (85), which is the acquisition means, performs wireless communication with the second wireless device and perform predetermined authentication on the communication partner, when the examinee 1 wearing the accessory 56 is carried into an ambulance (8) or the like and the responding second wireless device (56) is detected. When authentication is performed, the identification information 4 of the examinee that is stored in the storage unit (56) is transmitted from the second wireless device (56) to the first wireless device (85), enabling display of the health information 61 of the examinee 1 associated with this identification information 4. Accordingly, because the identification information 4 is acquired using the accessory 56 habitually worn by the examinee 1 such as a bracelet, the possibility of being able to acquire the identification information 4 is increased.

Note that in wearing the identification information 4 on the body, the accessory 56 is worn on the outside of the body of the examinee 1, but may also be embedded in the body; specifically, an IC chip that is used on animals.

There are also examinees such as children, elderly people and the like who will not possess the abovementioned information communication terminal 5, accessory 56, or the like. As such, a card 57 or a patient registration card 58 that the examinee 1 possesses may be used instead. A My Number card consisting of an IC card is given as an example of the card 57 but a magnetic card may also be used. In this case, the acquisition means is able to perform reading with card readers 812 and 814. When the card 57 is read, the personal computer 81 is able to refer to a table that is stored in advance in a server or the like other than the aggregation server of the management center 2, and easily read out the identification information 4 of the examinee 1 in the individual health information aggregation system from the number on the card 57.

On the other hand, the patient registration card 58 is the patient registration card of an arbitrary medical institution that the examinee 1 possesses, and the medical professional 9, by entering the information of the card with the personal computer 81, which is the acquisition means, is able to refer to a table that is stored in advance in a server or the like other than the aggregation server of the management center 2, and read out the identification information 4 of the examinee 1. In this individual health information aggregation system, originally, information relating to the examinee 1 gathered from a large number of medical institutions 3 is stored in association with one piece of identification information 4 that is allocated to each examinee 1, and thus information on the patient registration card 58 of each medical institution 3 is also included in the information relating to the examinee 1.

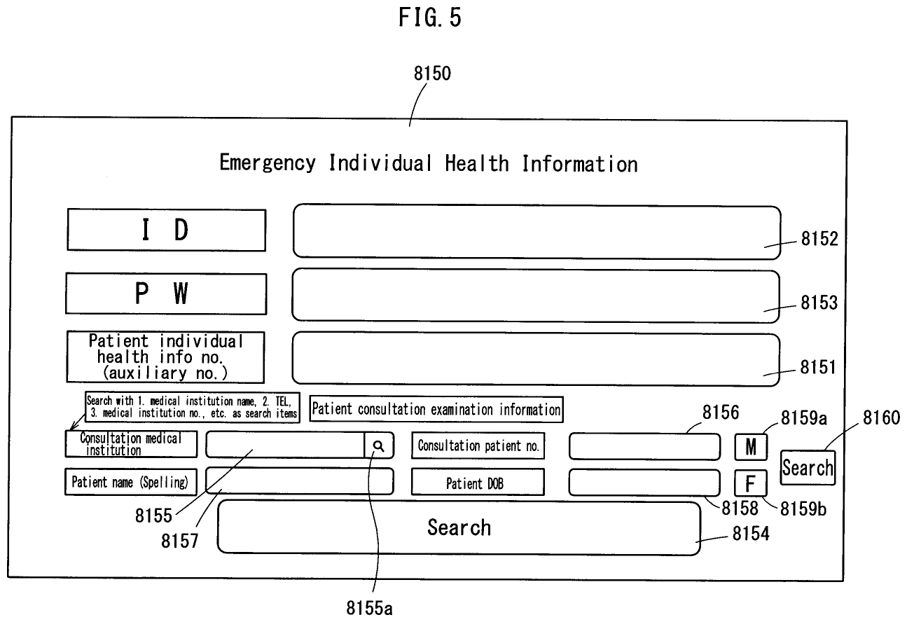

An example of an entry screen 8150 of the personal computer 81 in the case of using the information on this patient registration card 58 is shown in FIG. 5. FIG. 3 is similar to FIG. 5, with the same reference signs being given to corresponding portions, and description thereof being omitted. It should be noted that, on the entry screen 8150, fields 8155 to 8158 and buttons 8159a, 8159b and 8160 for entering the information on the patient registration card 58 of the examinee 1 are provided, in addition to the entry fields 8151 to 8153 and the login button 8154.

The entry field 8155 is a field for entering information on the medical institution that issued the patient registration card 58, or in other words, the medical institution where the examinee 1 receives consultation, and any of a name, a telephone number or a medical institution number need only be entered. In the case where a telephone number or a medical institution number is entered, the telephone number or medical institution number can be replaced with a name, by clicking a search button 8155a. The entry field 8156 is a field for entering a number (patient number, medical record number) of the examinee in the medical institution where he or she receives consultation, the entry field 8157 is a field for entering the spelling of the name of the examinee, and the entry field 8158 is a field for entering birth date of the examinee. The buttons 8159a and 8159b are buttons for clicking on to enter the gender of the examinee 1, and the search button 8160 is a button for searching a table that is stored in advance in a server or the like other than the aggregation server of the management center 2 for the identification information 4 of the entry field 8151 based on the patient registration card information.

By adopting this configuration, the identification information 4 can be easily read out with reference to the table from one card 57 or patient registration card 58 that the examinee 1 carries around with him or her, when the examinee 1 is in poor physical condition. Note that, in the case where the number on a personal identification card such as the patient registration card 58 is a two-dimensional barcode or even a so-called QR Code (registered trademark), manual entry with the keyboard 811 can be reduced, by reading the number with a barcode scanner.

Furthermore, even though there is a high possibility of the examinee (patient) 1 having the information communication terminal 5, the accessory 56 or the like on them, there is also a possibility of the examinee 1 forgetting to take their information communication terminal 5, accessory 56 or the like or not having one. In such cases, biometric authentication is effective in specifying an individual. That is, for example, a fingerprinting machine 88 which is collection means and a camera 89 which is photographing means are used. The fingerprinting machine 88 collects the fingerprint of a finger 101 of the examinee 1, and the personal computer 81 specifies an individual examinee 1, by collating the collected fingerprint with a database of fingerprints stored on a server or the like of the management center 2, which is a collation means, and the personal computer 81, which is readout means, reads out the personal information (identification information 4) of the specified examinee 1, and sets the read personal information in the entry field 8151 of the personal computer 81. Similarly, the personal computer 81 collates a portrait image taken with the camera 89 with a database of portrait images stored in another server or the like of the management center 2, which is collation means, and once an individual examinee 1 is specified, the personal computer 81, which is readout means, reads out the personal information (identification information 4) of the specified examinee 1, and sets the read personal information in the entry field 8151 of the personal computer 81. Acquisition means is constituted by this configuration.

As the fingerprint data, data from automatic passport gates, the police and the like can be utilized if available, but the majority will need to be newly registered with this system. In this respect, as face photo data, the large amount of data registered for driver's licenses, passports, My Number cards and the like can be useful if available. In the collation, personal information such as an address and a name is read out from the database based on fingerprints or a face photo, and that personal information is collated with a table of identification information 4 created by the mechanism of the individual health information aggregation system, that is, a table indicating which identification information 4 to allocate to whom, thus enabling identification information 4 to ultimately be read out based on fingerprints or a face photo. By adopting this configuration, as long as fingerprints or a face photo of the examinee 1 are registered in a database, the identification information 4 can be read out and medical records and the like made available, even if the examinee 1 does not have anything in particular on him or her and is in poor condition.

Next, a further method of utilizing this individual health information aggregation system after authentication of the examinee 1 in FIG. 4 will be described. When the health information 61 of an individual is obtained (downloaded) based on the identification information 4, a display screen 816 such as shown in FIG. 4 is displayed on the display screen 813 of the personal computer 81. The right-side field 8164 of the display screen 816, as aforementioned, is used for coordinating with the medical institution serving as the transportation destination, and, first, medical institutions serving as receiving destination candidates are displayed in candidate order in a list 8171. Also, the identification information 4 is displayed in an upper field 8172. The identification information 4 in the upper field 8172 and the health information 61 in left-side fields can be transmitted to the hospital designated in a field 8173 by clicking a send button 8174. This can be useful in helping the hospital prepare to receive the examinee 1.

In this individual health information aggregation system, a plurality of icons 8175 to 8178 for conveying the condition of the examinee 1 to the medical institution are also provided in the right-side field 8164 that is used for coordinating with the medical institution serving as the transportation destination. In icon 8175 portion, the result of a medical professional 9 such as a paramedic determining, over five levels, for example, the state of the examinee 1 determined through predetermined measures, is displayed, and by clicking on this icon 8175, the determination result is transmitted to the medical institution (henceforth, receiving medical institution) that is scheduled to ultimately receive the examinee 1 designated in the field 8173. In the case where the state of the examinee 1 is not easily conveyed with images or moving images which will be described later, this configuration enables the state of the examinee 1 to be quickly conveyed to the receiving medical institution.

Also, by clicking on the icon 8176, a handsfree calls are possible, enabling a medical professional 9 such as an emergency paramedic to seek instructions from a doctor in the receiving medical institution. Furthermore, by clicking on the icon 8177, a moving image of the accommodation place 8 shot with the aforementioned camera 89, which is image capturing means, or the like is transmitted to a display device 311 (refer to FIG. 1) of the receiving medical institution such as 31, for example. By clicking on the icon 8178, a still image of higher definition than the moving image, for example, in which the affected area or injury is shown in close-up is transmitted to the display device 311 of the medical institution 31, for example. The personal computer 81 serves as transmission means of these images and audio. Also, the management center 2 that transfers these images and audio to the display device 311 of the receiving medical institution 31 serves as relay means, but such relay is desirably performed by other relay means, without passing through the aggregation server of health information 61.

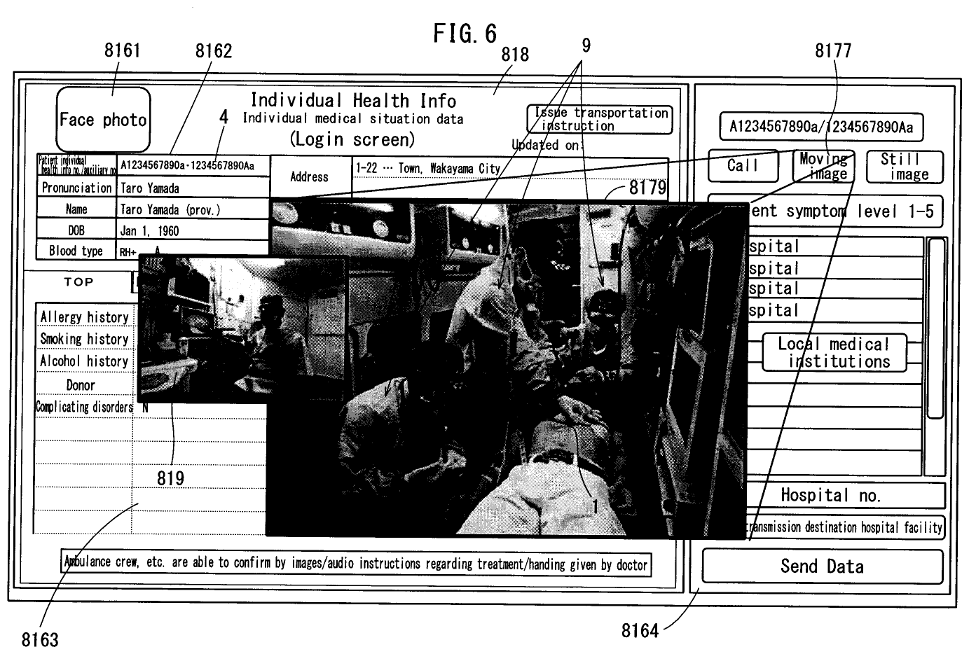

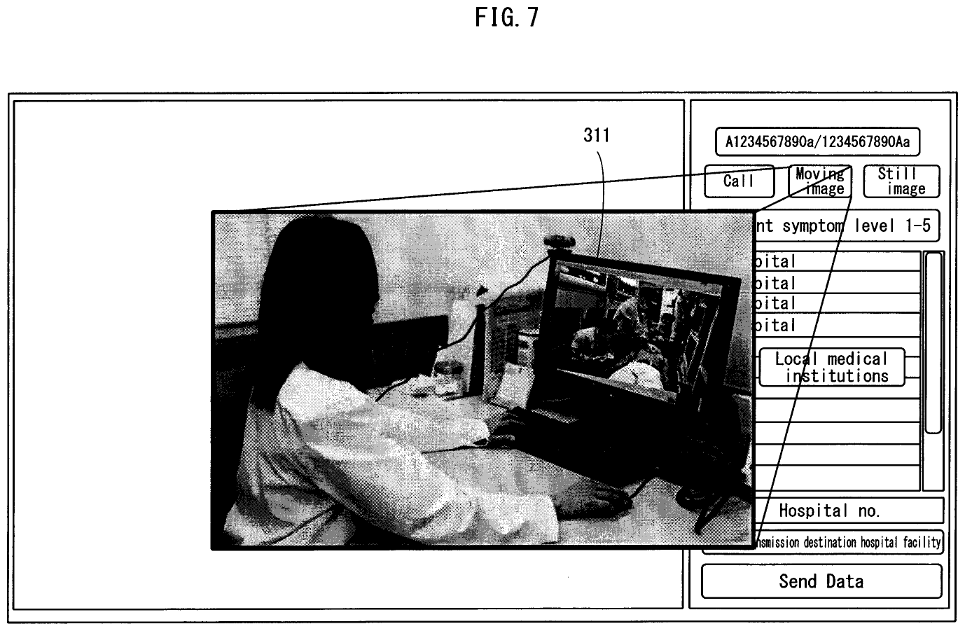

FIG. 6 shows an image of a display screen 818 of the personal computer 81 during live image transmission of the examinee 1 in an ambulance as the accommodation place 8. An image 819 of a doctor in the receiving medical institution 31 is also embedded in this screen 818 with a so-called teleconference technique. FIG. 7 shows an image during monitoring of the live image in the medical institution 31.

This enables appropriate preparations for receiving the examinee 1 to be made and the survival rate of the examinee 1 to be improved, by relaying live images of the examinee 1 and audio of the medical professional 9 in the accommodation place 8 to the medical institution 31, in addition to the health information 61 such as medical records. Also, with regard to the medical professional 9, complex tasks such as describing the symptoms and treatment to the receiving medical institution 31 that must be performed in parallel with treating the examinee 1 can be reduced. Also, by relaying at least audio from a doctor in the medical institution 31 to the personal computer 81 via a bidirectional line, instructions can be given regarding treatment or procedures and the situation to be confirmed in more detail. The survival rate of the examinee 1 can thereby be further improved.

The individual health information aggregation system of the present invention is an individual health information aggregation system that aggregates and holds health information for each of a plurality of examinees collected in a plurality of medical institutions in association with unique identification information assigned in advance to the examinee, and enables viewing of the health information of the examinee based on a predetermined rule, in response to a request of a medical professional and/or the examinee, including acquisition means for acquiring the identification information in an accommodation place accommodated where the examinee is accommodated when in poor physical condition, authentication means for performing authentication using an ID, a password or the like for a medical professional involved in treating the examinee to access (download) the health information of the examinee in the accommodation place, and display means for acquiring health information of the examinee associated with the identification information in the accommodation place when authentication is obtained by the authentication means, and displaying the acquired health information to the medical professional.

According to the above configuration, an individual health information aggregation system that aggregates and holds health information including current medical records and medication information for each of a plurality of examinees collected in a plurality of medical institutions and also a past clinical history or the like as appropriate in a management center or the like, and enables viewing of the health information of the examinee, in response to a request from a medical professional, the examinee or a family member or the like based on a predetermined rule is realized. As a result, this system is extremely effective for an examinee or a family member going to a new medical institution as an outpatient taking the health information of the examinee with him or her in order to get a second opinion or the like, for a local insurance society or medical association checking that there is no duplication of medication and no errors in the medical billing claim. Also, by using this system, troublesome and time-consuming tasks such as a getting referral, in order for the other hospital or clinical department to obtain the previous health information, can be dispensed with.

The predetermined rule for viewing the health information is for actions such as restricting the health information to a level viewable by a doctor, to a level viewable by a pharmacy, and to a level viewable by the examinee 1 himself or herself. The identification information is an ID number or the like that is allocated one to each examinee, and is not allocated for every medical institution unlike that on a patient registration card.

The present invention is configured such that medical professionals such as paramedics, doctors and nurses who perform first-aid measures are able to carry out appropriate measures and advance preparations utilizing health information, in the accommodation place of the examinee such as the inside of an ambulance or at a hospital serving as the transportation destination when the examinee is in poor physical condition, such as when transported in an emergency situation, for example, utilizing such an individual health information aggregation system. More specifically, identification information of the examinee needs to be acquired first, and acquisition means is provided. When this acquisition means acquires identification information in a manner that will be specifically described later, the medical professional carries out authentication as to whether he or she has the credentials to access (download) the health information of the examinee, using an ID, a password or the like, and, when authenticated, accesses (downloads) the health information and causes display means to display the health information.

Accordingly, by effectively using the health information of individuals that is aggregated and managed in a management center or the like, a medical professional is facilitated in taking appropriate action, even in an emergency, enabling the survival rate to be increased.

Also, in the individual health information aggregation system of the present invention, the examinee, at the point of desiring a new consultation as an outpatient or the like (at another hospital, etc.), via a network, has an information communication terminal having a dedicated application capable of accessing his or her own health information as appropriate, and this information communication terminal presents unique identification information assigned in advance to the examinee to the medical professional, when the examinee is in poor physical condition (when transported in an emergency situation, etc.).

According to the above configuration, the examinee, in the case of owning an information communication terminal capable of network connection such as a tablet terminal or a smartphone, installs the dedicated application of the individual health information aggregation system thereon, and, by being authenticated as an authorized user with the identification information or the like, the examinee becomes able to access his or her own health information as appropriate. Accordingly, the examinee can go for a new consultation as an outpatient or the like (at another hospital, etc.), for a second opinion or the like, taking the acquired health information with him or her. Given that such a dedicated application has been installed, the information communication terminal presents unique identification information assigned in advance to each examinee to the medical professional, when the examinee is in poor physical condition (when transported in an emergency situation, etc.).

Accordingly, acquisition of identification information by the acquisition means is facilitated. For example, it is sufficient to merely check the screen of a smartphone.

Furthermore, in the individual health information aggregation system of the present invention, there is a short-range wireless LAN spot in the accommodation place, a predetermined IP address common to this individual health information aggregation system is allocated to this wireless LAN spot, the information communication terminal has a dedicated application capable of accessing the health information of the examinee, and, upon a signal of a wireless LAN having the predetermined IP address being received, the dedicated application is activated, and presentation is performed by the identification information being displayed on a display screen or sent back with a signal of the wireless LAN.

According to the above configuration, the spot of a short-range wireless LAN such as a so-called Wi-Fi (registered trademark; IEEE-802.11 standard) or Bluetooth (registered trademark, IEEE-802.15 standard) is set so as to only be effective in the limited space of the accommodation place such as the inside of an ambulance or a treatment room, and, as a result of a signal of the predetermined IP address common to the system that was allocated to the wireless LAN spot being received, the dedicated application installed on the information communication terminal is activated, and the identification information is displayed on a display screen or sent back with a signal of the wireless LAN.

Accordingly, a medical professional is also able to reliably acquire identification information, in cases such as where the owner (examinee) is in poor condition, and the identification information cannot be retrieved by operating the mobile information terminal.

Also, in the individual health information aggregation system of the present invention, an auxiliary number capable of accessing the health information of the examinee similarly to the identification information is set in relation to the identification information, each examinee sets a user ID and password for accessing his or her own health information, using the information communication terminal, and the application of the information communication terminal sends back the auxiliary number together with the user ID and password with a signal of the wireless LAN.

According to the above configuration, each examinee is assigned in advance with the unique identification information for specifying the examinee by the system, and if this identification information is matched, the health information of the individual examinee that is aggregated in the system becomes viewable, based on the predetermined rule. On the other hand, generally, setting of a user ID and password will be required using an information communication terminal such as a smartphone, in order to login to any given system. As such, even with this system, in using the user ID and password, even though the health information of individual examinees is accessible with the identification information, an auxiliary number similar thereto is set, for example, and this auxiliary number is used on the wireless LAN, rather than the original identification information.

Accordingly, even though authentication by user ID and password is required, the information communication terminal automatically responds to a call from the wireless LAN, when the examinee is in poor physical condition, and even if the auxiliary number is intercepted by a third party, the identification information which is important personal information is not divulged, and personal information can be protected.

Furthermore, in the individual health information aggregation system of the present invention, the presentation of the unique identification information assigned in advance to the medical professional is triggered by a predetermined start operation performed on the information communication terminal by the medical professional.

Preferably, the information communication terminal already has (installed) a dedicated application capable of accessing the health information, and this dedicated application, as the predetermined start operation, recognizes a predetermined voice instruction by the medical professional though a so-called artificial intelligence (AI) speaker or speech recognition utilizing a search function, and the presentation is performed by displaying the identification information on a display screen.

According to the above configuration, in the case where the examinee owns an information communication terminal such as a tablet terminal or a smartphone, and the dedicated application of the individual health information aggregation system is installed thereon, a medical professional is able to link to the individual health information aggregation system through speech recognition software or search software with which the information communication terminal is provided as standard, and activate the dedicated application. When activated, the dedicated application performs presentation by the identification information of the owner (examinee) being displayed on a display screen, read out aloud, or the like.

Accordingly, a medical professional searches for identification information and becomes able to access health information, even if the owner (examinee) does not perform complex personal authentication, thus enabling identification information to be reliably acquired, even in a situation where the owner (examinee) is in poor condition and the identification information cannot be found out directly. Also, the dedicated application does not need to be left open all the time, enabling the power consumption of the information communication terminal to be reduced.

Also, in the individual health information aggregation system of the present invention, the identification information is transmitted to acquisition means from the information communication terminal by predetermined wireless communication, the identification information is automatically set to the acquisition means, and the identification information is transmitted between the acquisition means and the information communication terminal, after being encrypted using an encryption sequence that is changed at a predetermined timing.

According to the above configuration, in the case where a medical professional manually enters identification information displayed on an information communication terminal such as a smartphone, for example, to acquisition means such as a personal computer, in the accommodation place of the examinee such as the inside of an ambulance or a treatment room, in acquiring (setting) the identification information, the identification information does not leak outside of the accommodation place, but in the case where reduction of the load on the medical professional is achieved and the entry time is shortened, due to the identification information being transmitted by predetermined wireless communication such as wireless LAN and automatically set, the wireless communication may leak out of the accommodation place. Also, when a party having means to be able to pass authentication by authentication means such as the ID and password of a medical professional intercepts the identification information, medical records and other personal information of the examinee will be viewable.

As such, such undesirable leaking of personal information can be prevented, by encrypting the identification information, between the acquisition means and the information communication terminal, at a predetermined timing, such as a predetermined cycle, for example, with an encryption sequence that is changed each time.

Furthermore, in the individual health information aggregation system of the present invention, the accommodation place of the examinee is provided with a transmitter that is able to send notification of an emergency such as an earthquake at a transmission power capable of being received by an arbitrary information communication terminal that is in the accommodation place, via the network, and the transmitter sends the emergency notification in a dummy manner, in response to an operation by the medical professional, and by an information communication terminal that receives the notification reacting in a manner indicating that the incoming notification was received, the existence of the information communication terminal is indicated to the medical professional.

According to the above configuration, in the case where the medical professional does not know whether the owner (examinee) has an information communication terminal or where the information communication terminal is even if the examinee has one, notification of an emergency such as an earthquake is transmitted in a dummy manner at a power low enough to only be effective in the limited space of the accommodation place such as the inside of an ambulance or a treatment room, and the whereabouts of the mobile information terminal is confirmed by a reaction to the incoming notification.

Accordingly, even in situations such as where the owner (examinee) is in poor condition and the whereabouts of a mobile information terminal cannot be found out directly, the medical professional is able to reliably acquire the identification information.

Also, in the individual health information aggregation system of the present invention, the acquisition means is a first wireless device that searches (polls, probes) for a responding terminal using a mobile phone network or a wireless LAN network, in the accommodation place of the examinees such as an ambulance or a treatment room, the examinee wears an accessory that is habitually worn on the body such as a bracelet, and the accessory is provided with a storage unit that stores the identification information and a second wireless device that sends back the identification information to the first wireless device via the mobile phone network or the wireless LAN network, in response to the search.

According to the above configuration, acquisition of the identification information by the acquisition means is performed using an accessory such as a bracelet that is habitually worn by the examinee. More specifically, the acquisition means serves as a first wireless device that transmits a wireless signal that is only receivable in the narrow range of the accommodation place of the examinee such as an ambulance or a treatment room, and this first wireless device searches (probes) for a terminal that responds to the polling or the like, using a mobile phone network or a wireless LAN network. In situations such as where an examinee who is wearing the accessory is transported in an ambulance, the first wireless device, which is acquisition means, upon detecting the responding second wireless device, performs wireless communication with the second wireless device, and performs predetermined authentication on the communication partner. When authentication has been performed, identification information such as an ID of the examinee that is stored in the storage unit is transmitted from the second wireless device to the first wireless device, and display of the health information of the examinee associated with this identification information becomes possible.

Accordingly, identification information is acquired using an accessory habitually worn by the examinee such as a bracelet, increasing the possibility of being able to acquire the identification information.

Furthermore, in the individual health information aggregation system of the present invention, the acquisition means is configured to be provided with collection means for collecting fingerprints of the examinee, collation means for collating the collected fingerprints with a database and reading out personal information specifying an individual examinee, and readout means for reading out identification information of the examinee, with referral to a table of identification information registered in advance.

According to the above configuration, acquisition of identification information by the acquisition means involves reading out identification information of the examinee, through collating with a database of fingerprints registered in advance in passports, the police and the like. Specifically, personal information such as an address or a name is read out from a database based on fingerprints, and identification information is ultimately read out based on the fingerprints, by collating the personal information with a table of identification information created by the mechanism of the individual health information aggregation system, or in other words, with a table indicating which identification information to allocate to whom.

Accordingly, as long as the fingerprints of the examinee are registered in the database, identification information will be read out and medical records and the like will be viewable, even if the examinee has nothing in particular on him or her, and is in poor condition.

Also, in the individual health information aggregation system of the present invention, the acquisition means is configured to be provided with shooting means for shooting an image of a face of the examinee, collation means for collating the shot image with a database and reading out personal information specifying an individual examinee, and readout means for reading out identification information of the examinee, with referral to a table of identification information registered in advance.

According to the above configuration, acquisition of the identification information by the acquisition means involves reading out the identification information of the examinee collating with a database of shot images registered in advance on driver's licenses, passports, My Number, and the like. Specifically, personal information such as an address or a name is read out from a database based on a face image, and identification information is ultimately read out based on a face image, by collating this personal information with a table of identification information created by the mechanism of the individual health information aggregation system, or in other words, with a table indicating which identification information to allocate to whom.

Accordingly, as long as the face image of the examinee is registered in the database, identification information will be read out and medical records and the like will be viewable, even if the examinee has nothing in particular on him or her and is in poor condition.

Furthermore, in the individual health information aggregation system of the present invention, the acquisition means is a card reader, and the examinee possesses a card on which the identification information is recorded.

According to the above configuration, an IC card or the like registered in advance with My Number of the like can be utilized in the acquisition of identification information by the acquisition means.

Accordingly, a medical professional is able to easily read out identification information, by bringing the card in proximity to or in contact with the card reader, inserting the card into the card reader, or the like, and reading information.

Also, in the individual health information aggregation system of the present invention, the examinee possesses the patient registration card of an arbitrary medical institution, and the acquisition means is a table that stores a correspondence relationship between the patient registration card numbers of arbitrary medical institutions and the identification information of each examinee.

According to the above configuration, originally, in the individual health information aggregation system, information relating to each examinee gathered from a large number of medical institutions is stored in association with one piece of identification information allocated to that examinee, and thus the information relating to the examinee also includes information on the patient registration cards of various medical institutions.

Accordingly, with respect to the identification information of each examinee in the individual health information aggregation system, the patient registration card numbers of the various medical periods of that examinee are formulated as a table (tabulated), and the identification information can be easily read out with reference to this table from one of the patient registration cards that the examinee had on him or her, when the examinee is in poor physical condition.

Furthermore, in the individual health information aggregation system of the present invention, image capturing means for capturing an image in an accommodation place of the examinee such as an ambulance or a treatment room, transmission means for transmitting the identification information acquired by the acquisition means, information relating to authentication of an ID, password or the like of a medical professional acquired by the authentication means, and the captured image of the image capturing means, and relay means for relaying the captured image transmitted from the transmission means to a display device of a medical institution that is to receive the examinee are further provided.

According to the above configuration, an examinee can accept live images of the accommodation place of the examinee such as an ambulance or a treatment room can be relayed to a display device of the medical institution that is (scheduled) to (ultimately) receive the examinee.

Accordingly, the medical institution that is (scheduled) to receive the examinee is able to make appropriate preparations to receive the examinee, and the survival rate of the examinee can be improved. Also, with regard to medical professionals such as paramedics, complex tasks such as describing the symptoms and treatment to the receiving medical institution that must be performed in parallel with treating the examinee can be reduced.

Also, in the individual health information aggregation system of the present invention, reception means is provided in relation to the transmission means, and the relay means relays at least audio from the medical institution that is to receive the examinee to the reception means, via a bidirectional line.

According to the above configuration, at least audio is transmittable to the accommodation place of the examinee while viewing live images of the accommodation place of the examinee, thus enabling instructions to be given regarding treatment or procedures and the situation to be confirmed in more detail from the medical institution that is (scheduled) to receive the examinee.

Accordingly, the survival rate of the examinee can be further improved.

### INDUSTRIAL APPLICABILITY

With the individual health information aggregation system according to the present invention, in realizing efficient and appropriate treatment by using the aggregated health information of the examinees, in an accommodation place of an examinee such as inside an ambulance, when the examinee is in poor physical condition such as when being transported in an emergency situation, acquisition means acquires identification information of the examinee, and when it is authenticated in authentication means that a medical professional such as a paramedic has credentials to access the health information of the examinee, the health information of the examinee is displayed on display means. Accordingly, it becomes possible for the health information of individuals that is aggregated and managed to also be used in emergencies, facilitating medical professionals in taking appropriate action, enabling the survival rate to be improved, and being suitable for emergency care.

1. An individual health information aggregation system, comprising:
acquisition means for acquiring a unique identification information assigned in advance to an examinee, the acquisition means located in an accommodation place in which the examinee is accommodated when in poor physical condition;
authentication means for performing authentication of a medical professional involved in treating the examinee to access health information of the examinee, the health information collected in a plurality of medical institutions and associated with the identification information, in the accommodation place; and
display means in the accommodation place for acquiring the health information of the examinee associated with the identification information, after the authentication is performed by the authentication means, and displaying the acquired health information to the medical professional based on a predetermined rule.

2. The individual health information aggregation system according to claim 1, further comprising:
an information communication terminal associated with the examinee and configured to:
access the health information of the examinee via a network; and
present the identification information to the medical professional when the examinee is in the poor physical condition.

3. The individual health information aggregation system according to claim 2, further comprising:
a short-range wireless LAN spot provided in the accommodation place, the short-range wireless LAN spot associated with a predetermined IP address that is common to the individual health information aggregation, system; and
the information communication terminal comprising a dedicated application configured to access the health information of the examinee, and, upon a signal of a wireless LAN having the predetermined IP address being received, configured to activate and perform the presentation by at least one of displaying the identification information on a display screen of the information communication terminal or sending back the identification information with a further signal of the wireless LAN.

4. The individual health information aggregation system according to claim 3,
wherein an auxiliary number capable of accessing the health information of the examinee is associated with the identification information and is set in advance, and the examinee sets a user ID and a password for accessing the health information, using the information communication terminal, and
wherein the dedicated application of the information communication terminal is further configured to access the health information by sending the auxiliary number together with the user ID and password with a signal of the wireless LAN.

5. The individual health information aggregation system according to claim 2,
wherein the presentation of the unique identification information to the medical professional is triggered by a predetermined start operation performed on the information communication terminal by the medical professional.

6. The individual health information aggregation system according to claim 5, further comprising:
the information communication terminal comprising a dedicated application configured to access the health information, the dedicated application configured to activate upon recognizing a predetermined voice instruction by the medical professional through speech recognition, as the predetermined start operation, and further configured to perform the presentation by displaying the identification information on a display screen.

7. The individual health information aggregation system according to claim 5,
wherein the identification information is transmitted to the acquisition means from the information communication terminal via a predetermined wireless communication, and the identification information is automatically set in the acquisition means, and
wherein the identification information is transmitted between the acquisition means and the information communication terminal after being encrypted with an encryption sequence that is changed at a predetermined timing.

8. The individual health information aggregation system according to claim 2, further comprising:
a transmitter provided in the accommodation place, the transmitter configured to send in response to an operation by the medical professional an emergency notification via the network at a transmission power capable of being received by the information communication terminal when the information communication terminal is located in the accommodation place; and
the information communication terminal, upon receiving the emergency notification, configured to react in a manner that indicates that the emergency notification was received, existence of the information communication terminal is indicated to the medical professional.

9. The individual health information aggregation system according to claim 1,
wherein the acquisition means comprises a first wireless device configured to search for a responding terminal at least one of a mobile phone network or a wireless LAN network in the accommodation place of the examinee, further comprising:
an accessory worn by the examinee, the accessory comprising a storage unit that stores the identification information, and a second wireless device configured to send back the identification information to the first wireless device via one of the mobile phone network or the wireless LAN network, in response to the search.

10. The individual health information aggregation system according to claim 1,
wherein the acquisition means comprises:
collection means for collecting a fingerprint of the examinee;
collation means for collating the collected fingerprint with a database, and reading out personal information associated with the examinee based on the collated fingerprint and
readout means for reading out based on the personal information the identification information of the examinee, with referral to a table of identification information.

11. The individual health information aggregation system according to claim 1,
wherein the acquisition means comprises:
shooting means for shooting an image of a face of the examinee;
collation means for collating the shot image with a database, and reading out personal information associated with the examinee based on the collated image; and
readout means for reading out based on the personal information the identification information of the examinee, with referral to a table of identification information.

12. The individual health information aggregation system according to claim 1,
wherein the acquisition means comprises a card reader, and
the examinee possesses a card on which the identification information is recorded.

13. The individual health information aggregation system according to claim 1,
wherein the examinee possesses a patient registration card of one of a plurality of arbitrary medical institutions; and
the acquisition means comprises a table that stores a correspondence relationship between patient registration card numbers of the arbitrary medical institutions and the identification information of the examinee.

14. The individual health information aggregation system according to claim 1, further comprising:
image capturing means for capturing an image of the accommodation place;
transmission means for transmitting the captured image; and
relay means for relaying the captured image transmitted from the transmission means to a display device of a medical institution that is to receive the examinee.

15. The individual health information aggregation system according to claim 14, further comprising:
reception means,
wherein the relay means relays at least audio from the medical institution that is to receive the examinee to the reception means, via a bidirectional line.

16. The individual health information aggregation system according to claim 1, further comprising:
the acquisition means comprising a computing device configured to wirelessly receive, from a dedicated application executing on an information communication terminal associated with the examinee, the identification information encrypted by one of a plurality of encryption sequences stored on the information communication terminal and selected based on a predetermined cycle.

17. The individual health information aggregation system according to claim 1, further comprising:
the acquisition means comprising a wireless device, the wireless device configured to:
transmit a wireless signal only receivable within the accommodation place;
detect an accessory worn by the examinee that comprises a further wireless device responding to the signal; and
perform authentication with the further wireless device; and
receive the identification information from the further wireless device.

18. The individual health information aggregation system according to claim 1, further comprising:
a short-range wireless LAN spot provided in the accommodation place and interfaced with the acquisition means, the short-range wireless LAN spot associated with a predetermined IP address, the short-range wireless LAN spot configured to transmit a signal to an information communication terminal executing a dedicated application, wherein the dedicated application is activated upon receipt of the signal from the short-range wireless LAN spot with the predetermined IP address and provides the identification information for entry into the acquisition means.

19. The individual health information aggregation system according to claim 18, the short-range wireless LAN spot further configured to:
wirelessly receive the identification information from the information communication terminal; and
provide the received identification information to the acquisition means.

20. The individual health information aggregation system according to claim 1, the acquisition means further configured to:
send a predetermined message to a dedicated application executing on an information communication terminal associated with the examinee via a social networking service, wherein a receipt of the message activates the dedicated application; and
receive the identification information from the information communication terminal.
